(19) United States
(12) Patent Application Publication
(10) Pub. No.: US 2023/0355135 A1
(43) Pub. Date: Nov. 9, 2023

(54) INTELLIGENT GAIT ANALYZING APPARATUS

(71) Applicant: ASIA UNIVERSITY, Taichung (TW)

(72) Inventors: ZHI-REN TSAI, Taichung (TW); JING-PHA TSAI, Taichung (TW); CHIN-CHI KUO, Taichung (TW)

(21) Appl. No.: 17/736,065

(22) Filed: May 3, 2022

(51) Int. Cl. A61B 5/11; G16H 50/30; G16H 10/60; G06T 7/20

(52) U.S. Cl. CPC A61B 5/112; G16H 50/30; A61B 5/1128; G16H 10/60; G06T 7/20; G06T 2207/30196; G06T 2207/10016

(57) ABSTRACT

An intelligent gait analyzing apparatus comprises a first camera and an electronic device. The first camera captures a first video in which the patient walks and a second video in which the patient changes a posture from standing up to sitting down. A second camera may be used to capture a three-dimensional point cloud diagram of the patient to generate a chest skeleton and an abdomen skeleton. A 3D skeleton model and a moving diagram may be created based on the first video. The memory stores a long short-term memory model, a transformer machine learning model and an algorithm of modified gravity center. The graphic processor calculates a gravity center deviation based on the moving diagram, the chest skeleton and the abdomen skeleton. The graphic processor acquires a stride, a pace, a humpbacked value and a risk of falling.

### BACKGROUND OF THE INVENTION

### 1. Field of the Invention

The present invention relates to a gait analyzer. And particularly, the present invention relates to an intelligent gait analyzing apparatus which calculates a health score based on the health parameters (HP) as a stride, a pace, a humpbacked value (standing or walking body tilt angle), a risk of falling (a distance between a gravity center (x,y) and a center (x,y) of two feet), and a time length from the last sitting on the chair then to next standing up.

### 2. Description of the Related Art

As the health awareness rises and the trend of fitness is promoted, people are paying more attention to their health and spending time working out. Legs and feet are the fundamental of sport simply because healthy legs and feet are essential for the mobility in sports, such as running, swimming and playing badminton. If the legs and feet are disabled, such disability would restrain people from walking and playing the sport, and the enthusiasm about sport would be decreased. Hence, the monitoring the status of the capability of the legs and feet is quite important.

Currently, the conventional gait analyzer has been provided with the ability to analyze the walking behavior of people. The conventional gait analyzer utilizes a plurality of cameras to capture a walking image of one person and utilizes a plurality of sensors to sense the stress bearing by the joints and feet while the person is walking. The equipment for building up the conventional gait analyzer is complicated. The conventional gait analyzer is limited to analyzing the walking behavior of the person when the person walks, but it is difficult for the conventional gait analyzer to compare the current walking behavior with that in the past. If the conventional gait analyzer is utilized for a disabled person, a doctor needs to find the past walking behavior to evaluate the status of recovery of the disabled person and thus it reduces convenience.

Accordingly, the inventor of the present invention has designed an intelligent gait analyzing apparatus to overcome deficiencies in terms of current techniques so as to enhance the implementation and application in industries.

### SUMMARY OF THE INVENTION

According to the above problems, an objective of the present invention is to provide an intelligent gait analyzing apparatus builds a complete intelligent gait analyzing and estimating system through the capturing of cameras, the operation of algorithms and establishing of databases. A doctor is able to learn a past walking situation and a current walking situation from the intelligent gait analyzing apparatus of the present invention. The intelligent gait analyzing apparatus of the present invention is beneficial to the doctor to determine a better treatment to cure the patient.

For the abovementioned purpose, the present invention provides an intelligent gait analyzing apparatus. The intelligent gait analyzing apparatus comprises a chair, a first camera and an electronic device. The chair is arranged for a patient to sit and weigh. The first camera captures a first video in which the patient walks from a starting point to the chair and a second video in which the patient changes a posture from standing up to sitting on the chair. The electronic device is electrically connected to the first camera to receive the first video and the second video and is provided with a graphic processor and a memory. The memory stores an algorithm of gravity center and a calibration of skeleton coordinate. The graphic processor establishes a 3D skeleton model and a moving diagram based on the first video. The graphic processor acquires a stride, a pace, a risk of falling and a humpbacked value based on the moving diagram. The graphic processor utilizes the algorithm of gravity center to acquire an original gravity center based on the moving diagram to obtain a risk of falling and acquire a time length from the last sitting on the chair then to next standing up based on the second video. The graphic processor generates a health score by performing a weight function on the normalizing stride, the normalizing pace, the risk of falling based on the normalizing original gravity center, the humpbacked value and the time length from the last sitting on the chair then to next standing up.

Optionally, the moving diagram is a dynamic record recoding a motion of the 3D skeleton model corresponding to the patient in the first video and the graphic processor creates a plurality of coordinates on the 3D skeleton model.

Optionally, the graphic processor calculates the stride and the pace based on two coordinates corresponding to two feet in the moving diagram.

Optionally, the graphic processor utilizes the algorithm of gravity center to calculate the original gravity center based on a moving path of the plurality of coordinates in the moving diagram.

Optionally, the graphic processor utilizes the correction algorithm to calculate a modified gravity center based on the original gravity center and the thickness of the patient's thoracic surface and belly surface.

Optionally, the present invention further comprises a second camera. The second camera is electrically connected to the electronic device and captures a patient's 3D image of the patient. The electronic device creates a point cloud diagram based on the patient's 3D image.

Optionally, the graphic processor acquires a projection distance (or thickness) based on a boundary point of the point cloud diagram and utilizes the algorithm of gravity center to calculate a modified gravity center based on a moving path of the plurality of coordinates in the moving diagram and the projection distance.

Optionally, the present invention further comprises a workstation. The workstation is in networked communication with the electronic device and is provided with a backup database. The backup database stores a reference 3D skeleton model with a plurality of reference coordinates.

Optionally, the workstation transmits the reference 3D skeleton model to the electronic device and the graphic processor utilizes a long short-term memory (LSTM) model or a transformer machine learning model to calculate a variation between the plurality of coordinates and the plurality of reference coordinates or the health parameters (HP).

Optionally, the present invention further comprises a workstation. The workstation is in networked communication with the electronic device and is provided with a history database. The history database stores a history 3D skeleton model with a plurality of history coordinates.

Optionally, the workstation transmits the history 3D skeleton model to the electronic device, and the graphic processor utilizes a long short-term memory model or a transformer machine learning model to calculate a similarity score between the plurality of coordinates and the plurality of history coordinates or the health parameters (HP) related to abnormal symptom classes from an electric medical record. The graphic processor compares the similarity score with a threshold value.

Optionally, the electronic device comprises a first database and a second database. The first database stores the health score and the second database stores the 3D skeleton model and the moving diagram.

Optionally, the time length from the last sitting on the chair then to next standing up is determined by the graphic processor calculating a time difference for a head and a pelvis of the body segments moving from a first coordinate of the head and the pelvis representing the patient is standing up and a second coordinate of the head and the pelvis representing the patient is sitting on the chair.

Optionally, the present invention further comprises a pathway. The pathway is provided with the starting point and is adjacent to the chair to provide the patient to walk from the starting point to the chair.

Optionally, the present invention further comprises a sensor. The sensor is electrically connected to the electronic device and senses an identification card of the patient to acquire an identification number. The electronic device receives the identification number.

Optionally, the present invention further comprises a medical care platform. The medical care platform is in networked communication with the electronic device to receive the identification number and the health score. The medical care platform transmits an electronic medical record to the electronic device.

In accordance with the above description, the intelligent gait analyzing apparatus of the present invention utilizes a first camera to capture a video in which the patient walks, sits down and stands up. Subsequently, the graphic processor acquires the stride, the pace, the humpbacked value, the risk of falling and the time length from the last sitting on the chair then to next standing up based on the video and utilizes the algorithm of gravity center to acquire an original gravity center. At last, the graphic processor calculates the health score by performing a weight function on the stride, the pace, the humpbacked value, the risk of falling and the time length from the last sitting on the chair then to next standing up. Doctor estimates recovery situation and muscle endurance of the patient.

### BRIEF DESCRIPTION OF THE DRAWINGS

FIG. 1 is a configuration diagram of the intelligent gait analyzing apparatus according to the first embodiment of the present invention.

FIG. 2 is a block diagram of the intelligent gait analyzing apparatus according to the first embodiment of the present invention.

FIG. 3 is an operation process diagram of the intelligent gait analyzing apparatus according to the first embodiment of the present invention.

FIG. 4 is a 3D skeleton model diagram of the intelligent gait analyzing apparatus according to the first embodiment of the present invention.

FIG. 5 is a moving diagram of the intelligent gait analyzing apparatus according to the first embodiment of the present invention.

FIG. 6 is a normalized moving diagram of the intelligent gait analyzing apparatus according to the first embodiment of the present invention.

FIG. 7 is a diagram showing depth variation of a pelvis according to the first embodiment of the present invention.

FIG. 8 is a block diagram of the intelligent gait analyzing apparatus according to the second embodiment of the present invention.

FIG. 9 is a point cloud diagram of the intelligent gait analyzing apparatus according to the second embodiment of the present invention.

FIG. 10 is a 3D skeleton model diagram of the intelligent gait analyzing apparatus according to the second embodiment of the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” comprises any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise” and variations such as “comprises” or “comprising”, will be acknowledged to imply the inclusion of stated elements but not the exclusion of any other elements.

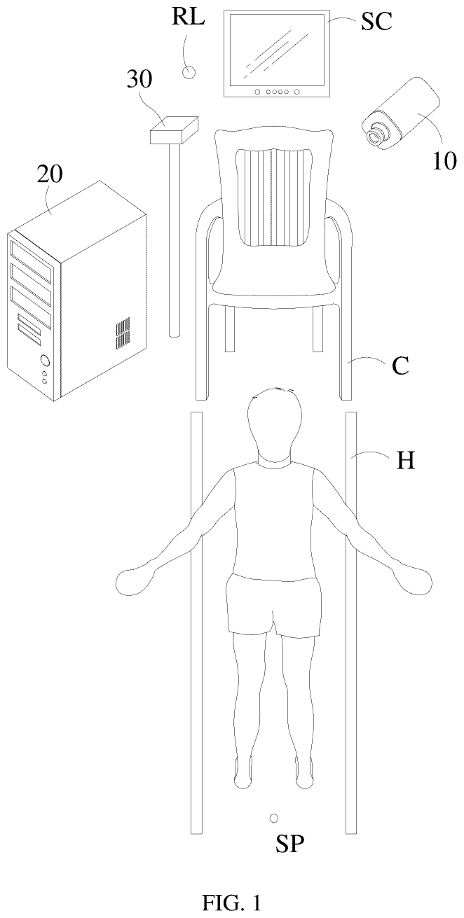

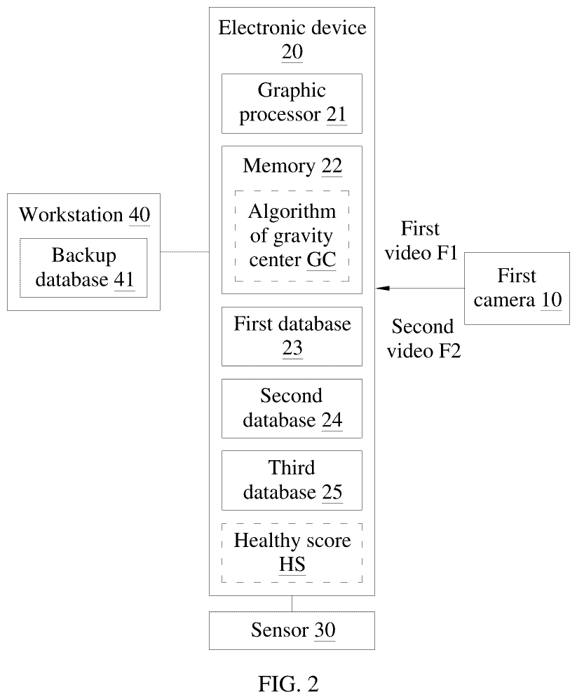

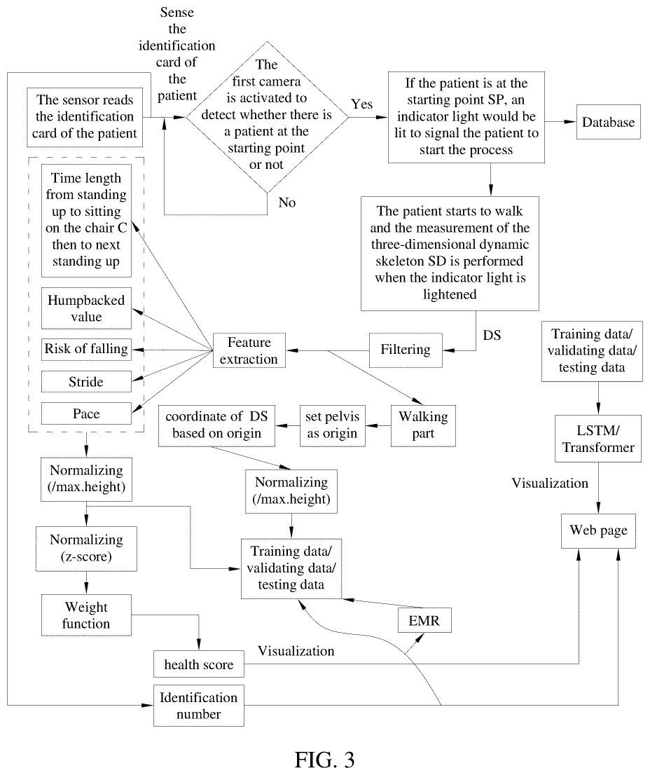

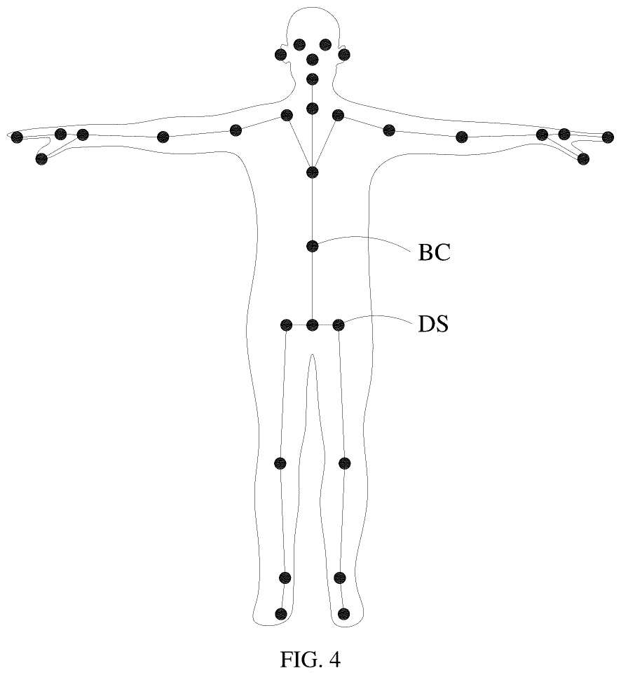

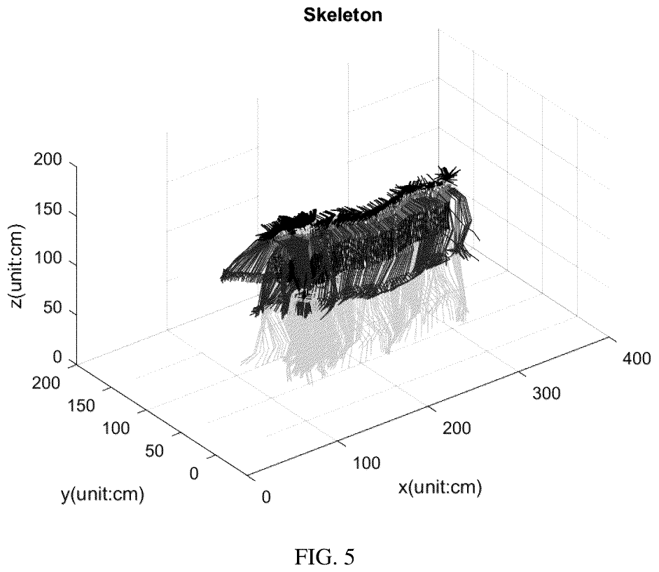

Please refer to FIG. 1, FIG. 2 and FIG. 3, which are respectively a configuration diagram of the intelligent gait analyzing apparatus according to the first embodiment of the present invention, a block diagram of the intelligent gait analyzing apparatus according to the first embodiment of the present invention, and an operation process diagram of the intelligent gait analyzing apparatus according to the first embodiment of the present invention. As shown in FIG. 1 and FIG. 2, the intelligent gait analyzing apparatus of the present invention may be provided with a chair C, a first camera 10 and an electronic device 20. The chair C is arranged for a patient to sit. The first camera 10 captures a first video F1 in which the patient walks from a starting point SP to the chair C and a second video F2 in which the patient changes a posture from standing up to sitting on the chair C. The electronic device 20 is electrically connected to the first camera 10 to receive the first video F1 and the second video F2, and the electronic device 20 is provided with a graphic processor 21 and a memory 22. The memory 22 stores an algorithm of gravity center GC. The graphic processor 21 establishes a 3D skeleton model DS and a moving diagram MD (as shown in FIG. 4 and FIG. 5) based on the first video F1. The moving diagram MD is a dynamic record recoding a motion of the 3D skeleton model DS corresponding to the patient in the first video F1, and the graphic processor 21 creates a plurality of coordinates BC (as shown by FIG. 4) to define a plurality of body segments of the 3D skeleton model DS. The graphic processor 21 utilizes the algorithm of gravity center GC to acquire an original gravity center based on the moving diagram MD. The graphic processor 21 acquires a stride, a pace, a risk of falling and a humpbacked value based on the moving diagram MD. The graphic processor 21 acquires a time length from the last sitting on the chair then to next standing up based on the second video F2. The graphic processor 21 generates a health score HS by normalizing and performing a weight function on the stride, the pace, the risk of falling the humpbacked value and the time length from the last sitting on the chair then to next standing up.

It is worthy to note that the maximum and the minimum values of the stride, the maximum and the minimum values of the pace, the maximum and the minimum values of the risk of falling, the maximum and the minimum values of the humpbacked value and the maximum and the minimum of the time length from the last sitting on the chair then to next standing up may be provided to the graphic processor 21 prior to the evaluation of the patient. The maximum and the minimum values of the stride, the pace, the risk of falling, the humpbacked value and the time length from the last sitting on the chair then to next standing up are respectively scaled proportionally from 0 to 100 for normalization of the measured values. The graphic processor 21 generates scores of the measured values of the stride, the pace, the risk of falling, the humpbacked value and the time length from the last sitting on the chair then to next standing up in proportion to each of the range of the respective maximum and the minimum values. Doctors may additionally customize weightings of the stride, the pace, the risk of falling, the humpbacked value and the time length from the last sitting on the chair then to next standing up. The graphic processor 21 performs a weight function on the stride, the pace, the risk of falling, the humpbacked value and the time length from the last sitting on the chair then to next standing up according to the weightings in order to acquire the overall combined health score HS.

The electronic device 20 may include a first database 23, a second database 24 and a third database 25. The first database 23 stores the health score HS. The second database 24 stores the 3D skeleton model DS and the moving diagram MD. The third database 25 stores the identification number. The first camera 10 may be a Kinect sensor and the electronic device 20 may be a desktop computer or a laptop computer. The electronic device 20 may be other devices with the function of processing, but is not limited thereto. It is worthy to mention that Azure Kinect may be used in the present invention as the first camera, for the high hardware performance of its GPU power, more data points in the point cloud may be provided for precision measurement, and a processing speed thereof may be increased dramatically, up to 30 times of speed, while comparing to conventional Kinect devices.

In the present embodiment, a pathway H, a sensor 30 and a workstation 40 may be arranged. The pathway H comprises a starting point SP and is adjacent to the chair C to provide the patient to walk from the starting point SP to the chair C. The handrails may be disposed on two sides of the pathway H according to the actual need, which may be beneficial for a disabled person to walk. For example, a size of the pathway H is 1.5 m in length and 0.6 m in width. The sensor 30 is electrically connected to the electronic device 20 and senses an identification card of the patient to acquire an identification number. The electronic device 20 receives the identification number. For example, the sensor 30 may be a radio frequency identification (RFID) sensor and the identification card of the patient may include an RFID tag. The workstation 40 may be in networked communication with the electronic device 20 and may be provided with a backup database 41. The backup database 41 may store a plurality of reference 3D skeleton models, each of which may include a plurality of reference coordinates defining a plurality of body segments. A plurality of the reference 3D skeleton models may be divided into a plurality of groups. Each group may have at least one reference 3D skeleton model and the plurality of reference coordinates thereof. The workstation 40 may be a server or a super computer. The workstation 40 may be other types of the electronic devices, but is not limited thereto.

Please refer to FIG. 3, which is an operation process diagram of the intelligent gait analyzing apparatus according to the first embodiment of the present invention. As shown in FIG. 3, in collaboration with FIG. 1, FIG. 2 and FIG. 4, the use and operation of the intelligent gait analyzing apparatus are illustrated as follows: the patient may firstly insert the identification card of the patient into a receiving slot of the sensor 30 or may place the identification card of the patient on the sensing face of the sensor 30. The sensor 30 would retrieve the identification number of the identification card of the patient and transmit the identification number to the third database 25 of the electronic device 20. The first camera 10 may then be activated to detect whether the patient is at the starting point SP or not. If the patient is at the starting point SP, an indicator light would be lit to signal the patient to start the process. If the patient is not at the starting point SP, the indicator light is not lit and the first camera would repeat the detection.

When the indicator light is lit, the patient may start walking toward the chair C along the pathway H, the first camera 10 may then capture the walking progress of the patient as the first video F1. When the patient changes a posture from standing up to sitting on the chair C, the first camera 10 would capture the progress of changing posture of the patient as a second video F2. The first camera 10 transmits the first video F1 and the second video F2 to the electronic device 20. When the patient sits on the chair C, the chair C is able to measure weight of the patient and a screen SC displays weight of the patient. Afterwards, the graphic processor 21 may construct the 3D skeleton model DS and the moving diagram MD based on the first video F1. The graphic processor 21 would acquire the stride, the pace and the height variation based on the moving diagram MD. The graphic processor 21 may utilize the algorithm of gravity center GC to generate the original gravity center based on a moving path of the plurality of coordinates BC of the 3D skeleton model DS of the moving diagram MD. The graphic processor may further utilize the correction algorithm to calculate a variation of gravity center based on the original gravity center and the variation of the patient. The graphic processor 21 acquires a time length from the last sitting on the chair then to next standing up based on the second video F2. The graphic processor 21 generates a health score HS by performing a weight function on the scores of the measured stride, the pace, the humpbacked value, the risk of falling and the time length from the last sitting on the chair then to next standing up. Hereafter, the graphic processor 21 transmits the health score HS to the first database 23 and the backup database 41 and transmits the 3D skeleton model DS and the moving diagram MD to the second database 24 and the backup database 41.

And then, the workstation 40 may select the reference 3D skeleton model corresponding to the received 3D skeleton model DS and the plurality of reference coordinates thereof from the backup database 41. The workstation 40 may transmit the reference 3D skeleton model corresponding to the received 3D skeleton model DS and the plurality of reference coordinates thereof to the graphic processor 21. The graphic processor 21 would compare the received 3D skeleton model DS and the plurality of coordinates BC thereof with the corresponding reference 3D skeleton model and the plurality of reference coordinates thereof by the long short-term memory model or the transformer machine learning model to acquire a difference. The graphic processor 21 classifies the difference obtained from the long short-term memory model or the transformer machine learning model as one of the plurality of groups. The graphic processor 21 may then transmit the difference, the corresponding group, the health parameters (HP), the received 3D skeleton model DS and the plurality of coordinates BC thereof to the backup database 41. The backup database 41 may store the received 3D skeleton model DS, the health parameters (HP) and the plurality of coordinates BC thereof into the corresponding group. Alternatively, the graphic processor 21 may transmit the received 3D skeleton model DS, the health parameters (HP) and the plurality of coordinates BC thereof to the workstation 40. The workstation 40 may select the reference 3D skeleton model corresponding to the received 3D skeleton model DS, the health parameters (HP) and the plurality of reference coordinates thereof from the backup database 41. The processor of the workstation 40 may compare the received 3D skeleton model DS, the health parameters (HP) and the plurality of coordinates BC thereof with the corresponding reference 3D skeleton model and the plurality of reference coordinates thereof by the long short-term memory model or the transformer machine learning model to acquire the difference. The processor of the workstation 40 classifies the difference obtained from the long short-term memory model or the transformer machine learning model as one of the plurality of groups. The processor of the workstation 40 may store the received 3D skeleton model DS, the health parameters (HP) and the plurality of coordinates BC thereof into the corresponding group.

In another embodiment, the workstation 40 may be provided with a history database. The history database may store a history 3D skeleton model with a plurality of history coordinates or the health parameters (HP) related to abnormal symptom classes from the electric medical record ER. The history 3D skeleton model may be a 3D skeleton model generated and stored when the patient used the present invention in the past. The workstation 40 may select the history 3D skeleton model corresponding to the received 3D skeleton model DS, the health parameters (HP) and the plurality of history coordinates thereof from the history database 41. The workstation 40 may then transmit the history 3D skeleton model corresponding to the received 3D skeleton model DS, the health parameters (HP) and the plurality of history coordinates thereof to the graphic processor 21. The graphic processor 21 would calculate a similarity score between the plurality of received coordinates BC, the health parameters (HP) and the plurality of history coordinates by a transformer machine learning model or a long short-term memory model and the graphic processor 21 may then compare the similarity score with a threshold value. If the graphic processor 21 determines that the similarity score is greater than the threshold value, such similarity would indicate that the feet situation of the patient substantially unchanged prior to the treatment, and the doctor may modify the treatment of the patient based on the similarity score. If the graphic processor 21 determines that the similarity score is less than the threshold value, which would indicate that feet situation of the patient is in the process of recovery, and the doctor may further evaluate recovery situation of the patient based on the stride, the pace, the humpbacked value, the risk of falling, the time length from the last sitting on the chair then to next standing up and the similarity score. The doctor may determine whether the patient has a hunchback posture due to illness or not based on the height variation. The dynamic graph convolution neural network may then perform machine learning and more instantly calculate the similar score.

In other embodiment, the graphic processor 21 may extract the features from the first video F1 and the second video F2 to obtain the time length from the last sitting on the chair C then to next standing up, the humpbacked value, the risk of falling. The above features may combine with the stride and the pace to calculate the health score by performing the normalizing (with max height and z-score) and the weight function process. In some embodiments, the hand grip strength or the foot pressure point value can be added to determine the health score.

Finally, the workstation 40 may transmit the received 3D skeleton model DS, the health parameters (HP), the plurality of history coordinates thereof and the corresponding difference to the cloud platform, and the electronic device 20 may also transmit the health score HS to the cloud platform. The cloud platform may then transmit the received 3D skeleton model DS, the health parameters (HP), the plurality of history coordinates thereof, the corresponding variation and the health score HS to the screen DS. The screen SC may consequently display the received 3D skeleton model DS, the health parameters (HP), the plurality of history coordinates thereof, the corresponding variation and the health score HS to the patient.

Besides, the present invention may not only measure the 3D skeleton model DS and analyzes the gait, but also serve as a health score and variation automatic marking system for the other medical purposes, such as an evaluation system of rehabilitation medical assistive device, an evaluation system of sarcopenia behavior, an evaluation system of clinical trial of IPS and research of the other health score analysis or evaluation related to the body motion.

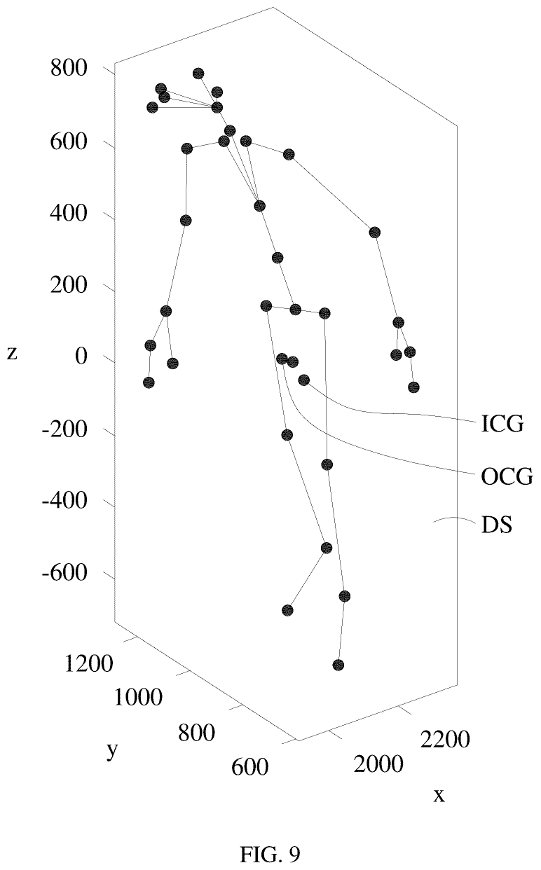

Please refer to FIG. 4, which is a 3D skeleton model diagram of the intelligent gait analyzing apparatus according to the first embodiment of the present invention. As shown in FIG. 4, in collaboration with FIG. 1 and FIG. 2, the graphic processor 21 may construct the 3D skeleton model DS and the moving diagram MD based on the first video F1. If the first camera 10 is a Kinect sensor, the first camera 10 (Kinect) has thirty-two coordinates BC in body tracking. Namely, the first camera 10 may track thirty-two body parts of the patient. However, the computation of thirty-two coordinates BC may be too intense for the graphic processor 21. The essay of “A method for measuring the movement of Gravity center of body of golf swing based on Kinect Sensor” (Xiangmin Wang, IJECS Volume 7 Issue 7 Jul. 2018 Page No. 24168-24173) is cited. The essay collects fifteen mass center positions and weight distribution of fifteen body parts of males and females. Accordingly, the thirty-two coordinates of the Kinect sensor may be simplified and decreased to fifteen coordinates. The algorithm of gravity center thereof may be modified to derive two skeletons (chest skeleton and abdomen skeleton) which is used in the proposed algorithm of the modified gravity center (as shown by FIG. 9) in the present invention. The graphic processor 21 may then calculate the original gravity center OCG and the modified gravity center ICG (as shown by FIG. 9) accordingly.

Please refer to FIG. 5, which is a moving diagram of the intelligent gait analyzing apparatus according to the first embodiment of the present invention. As shown by FIG. 5, in collaboration with FIG. 1 to FIG. 3, x-axis is parallel to a length direction of the pathway H, y-axis is parallel to a width direction of the pathway H and z-axis is parallel to a height direction of the patient. The pathway H may be divided into several parts and the important articulation point diagram of the moving diagram MD may be divided into each sections. The graphic processor 21 may establish the 3D skeleton model DS and the plurality of coordinates BC thereof according the first video F1. The walking progress in the four parts of the pathway H of the patent may be stored in respectively four sections S1-S4 of the motion diagram MD with the established 3D skeleton model DS. The graphic processor 21 selects the 3D skeleton model DS and the plurality of coordinates BC thereof corresponding to the maximum stride from the sections of the motion diagram MD and transmits the 3D skeleton model DS and the plurality of coordinates BC thereof corresponding to the maximum stride to the backup database 41 as the reference 3D skeleton model and the plurality of reference coordinates thereof. The graphic processor 21 utilizes the long short-term memory model or the transformer machine learning model to compare the plurality of 3D skeleton models DS and the plurality of coordinates BC thereof recorded in the moving diagram MD with the reference 3D skeleton model and the plurality of reference coordinates thereof to acquire the plurality of differences. The graphic processor 21 classifies the plurality of differences and the plurality of 3D skeleton models DS and the plurality of coordinates BC thereof corresponding to the plurality of differences. The graphic processor 21 stores the plurality of classified 3D skeleton models DS and the plurality of classified coordinates BC thereof in the backup database 41. Besides, the graphic processor 21 marks a maximum stride, a maximum pace, a maximum risk of falling, a maximum humpbacked value, and a shortest time length from the last sitting on the chair then to next standing up in the corresponding 3D skeleton model DS and transmits the marked 3D skeleton model DS to the cloud platform. The cloud platform transmits the marked 3D skeleton model DS to the screen SC for displaying the marked 3D skeleton model DS to the patient.

It is worthy to be mentioned that the graphic processor 21 illustrates curves related to the maximum stride per day, the maximum pace per day, the maximum risk of falling per day, the maximum humpbacked value per day and the shortest time length from the last sitting on the chair then to next standing up per day after the patient has used the present invention within a period of one month. It is beneficial for the doctor to observe the recovery situation of the patient. By using the long short-term memory model or the transformer machine learning model, the prediction of the behavior of the stroke patients may have an accuracy rate of about 90%.

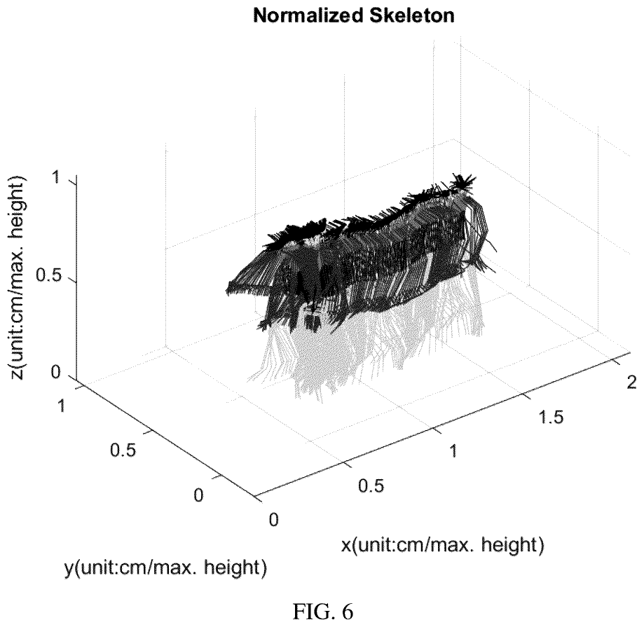

Please refer to FIG. 6, which is a normalized moving diagram of the intelligent gait analyzing apparatus according to the first embodiment of the present invention. FIG. 6 is the normalized moving diagram of FIG. 5. When conducting the calculation process, the measured data can be used to generate the health score. However, the different patients may have different heights. The measured data will be varied because of the different heights. In order to eliminate these differences reference, the measured height values are normalized by the max height to obtain the normalized diagram. The reference 3D skeleton model or the history 3D skeleton model is also stored by the normalized value. When the electronic device 20 conduct the comparing and calculating processes, there will be no error in judgment due to height differences from the patients.

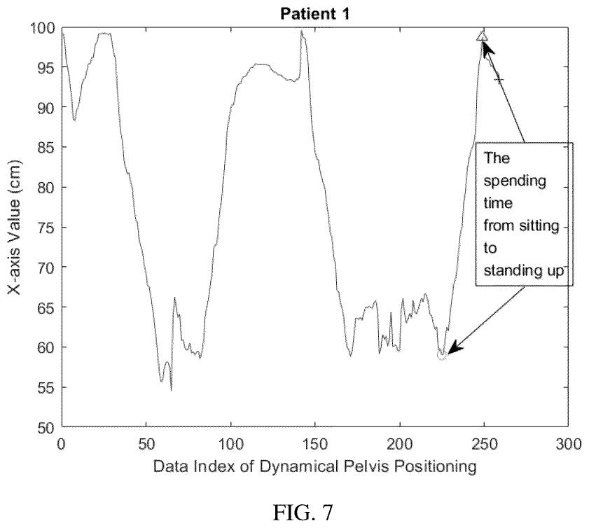

Please refer to FIG. 5 and FIG. 7, which is a diagram showing depth variation of a pelvis according to the first embodiment of the present invention. As shown by FIG. 7, in collaboration with FIG. 1 and FIG. 5, the height of a head and the depth of a pelvis vary when the patient changes a posture from standing up to sitting down (during the record of the second video F2). The axis of the height variation corresponds to z-axis of FIG. 5. During the measurement, the patient is required to change the posture from standing up to sitting down at least once, that is, from standing up to sitting down, then from sitting down to standing up. Such cycle of changing posture is required to be completed as fast as the patent is able to. A z coordinate representing the head, hereinafter, the head z coordinate and the depth x coordinate, of the patient vary when the patient changes a posture from standing up to sitting down and vice versa. Consequently, a first sitting point FS and a second sitting point SS may be defined respective at the ending points of the descending curves as illustrated in FIG. 7. For example, when the patient changes from standing up to sitting down for the first time, the head z coordinate and the pelvis x coordinate would start to decrease. When the patent is actually sitting on the chair, the head z coordinate and the pelvis x coordinate would stop decrease and remain relatively stable. The graphic processor 21 may therefore finds the first sitting point FS accordingly. Then, when the patient starts to stand up, the head z coordinate and the pelvis x coordinate would start to increase until the patent is actually standing up. Then, again, when the patent changes from standing up to sitting down for the second time, the head z coordinate and the pelvis x coordinate would start to decrease. When the patent is actually sitting on the chair, the head z coordinate and the pelvis x coordinate would stop decrease and remain relatively stable. The graphic processor 21 may therefore finds the second sitting point SS accordingly. The time difference between the last sitting down point FS and the standing up point SS may be calculated by the graphic processor 21 as the time length from the last sitting on the chair then to next standing up.

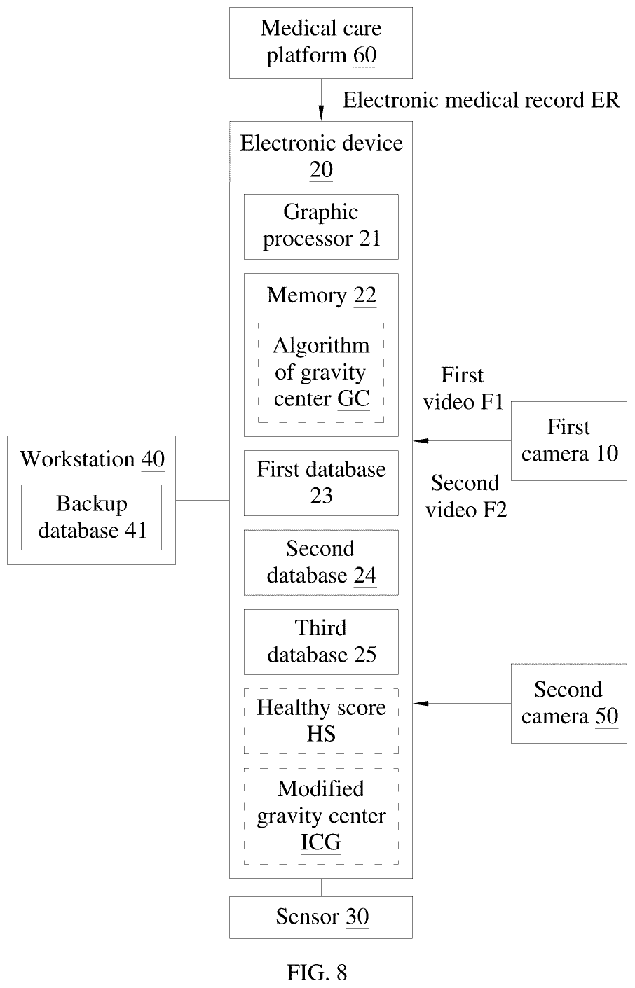

Please refer to FIG. 1 to FIG. 10, which are respectively the block diagram of the intelligent gait analyzing apparatus according to the second embodiment of the present invention, the point cloud diagram of the intelligent gait analyzing apparatus according to the second embodiment of the present invention and the 3D skeleton model diagram of the intelligent gait analyzing apparatus according to the second embodiment of the present invention. As shown in FIG. 8, the intelligent gait analyzing apparatus may be provided with the chair C, the first camera 10, the electronic device 20, the pathway H, the sensor 30, the workstation 40, a second camera 50 and a medical care platform 60. The chair C, the first camera 10, the electronic device 20, the pathway H, the sensor 30, the workstation 40 of the second embodiment are similar to these of the first embodiment and would not be described again.

The second camera 50 is electrically connected to the electronic device 20 and captures patient images of the patient. The medical care platform 60 is in networked communication with the electronic device 20 to receive the identification number and the health score HS. The medical care platform 60 transmits the electronic medical record ER to the electronic device 20 based on the identification number.

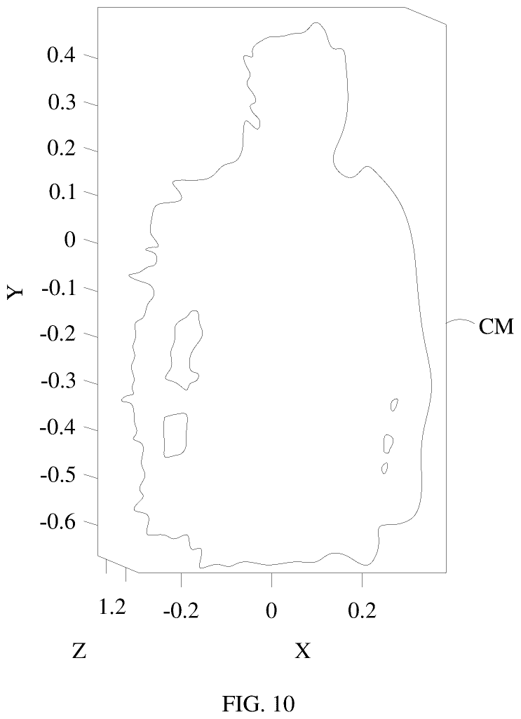

As shown in FIG. 9 and FIG. 10, in collaboration with FIG. 8, the second camera 50 captures the patient images and transmits the patient images to the electronic device 20. The graphic processor 21 creates the point cloud diagram CM based on the patient images. The graphic processor 21 sets up different projection distances and different boundary points from the point cloud diagram CM based on pigeon chest and protuberant abdomen of males and females. The graphic processor 21 utilizes the algorithm of modified gravity center to calculate the modified gravity center ICG based on the moving path of the plurality of coordinates BC of the 3D skeleton model DS in the moving diagram MD (as shown in FIG. 5), the projection distance and the boundary point. The graphic processor 21 utilizes the algorithm of gravity center GC to calculate the original gravity center OCG based on the moving path of the plurality of coordinates BC of the 3D skeleton model DS in the moving diagram MD and marks the original gravity center OCG on the 3D skeleton model DS.

If the patient is the male with pigeon chest, the graphic processor 21 obtains a chest part (i.e. chest skeleton) from the point cloud diagram CM and calculates the projection distance to ground (i.e. the pathway H) between a midpoint of the chest part and the midpoint of a rising edge of the chest part (i.e. a joint point between chest and neck). The front foot of the patient is regarded as the boundary point. The boundary point and the projection distance are input to the algorithm of modified gravity center. The graphic processor 21 calculates the modified gravity center ICG based on the moving path of the plurality of coordinates BC of the 3D skeleton model DS in the moving diagram MD, the projection distance and the boundary point and marks the modified gravity center ICG on the 3D skeleton model DS. If the patient is the female with pigeon chest, the calculation of the modified gravity center ICG about the female with pigeon chest is similar to the calculation of the modified gravity center ICG about the male with pigeon chest except the projection distance and the boundary point of the female are different these of the male.

If the patient is the male with protuberant abdomen, the graphic processor 21 obtains an abdomen part (i.e. abdomen skeleton) from the point cloud diagram CM and calculates the projection distance to ground (i.e. the pathway H) between a midpoint of the abdomen part and the midpoint of a rising edge of the chest part (i.e. a joint point between chest and neck). The front foot of the patient is regarded as the boundary point. The boundary point and the projection distance are input to the algorithm of modified gravity center. The graphic processor 21 calculates the modified gravity center ICG based on the moving path of the plurality of coordinates BC of the 3D skeleton model DS in the moving diagram MD, the projection distance and the boundary point and marks the modified gravity center ICG on the 3D skeleton model DS. If the patient is the female with pigeon chest, the calculation of the modified gravity center ICG about the female with pigeon chest is similar to the calculation of the modified gravity center ICG about the male with pigeon chest except the projection distance and the boundary point of the female are different these of the male.

Please refer to FIG. 8 again, the medical care platform 60 transmits the electronic medical record ER to the electronic device 20 based on the identification number. The electronic medical record ER may include the heart rate, the breath rate, the body temperature and the blood pressure of the patient. The heart rate, the breath rate, the body temperature and the blood pressure of the patient and daily habits (e.g. smoking or chewing betel nuts) of the patient are normalized. The graphic processor 21 restart to perform a weight function on physiological parameters of the electronic medical record ER, the stride, the pace, the humpbacked value, the risk of falling and the time length from the last sitting on the chair then to next standing up on the chair to acquire a new health score. The electronic device 20 transmits the new health score to the medical care platform 60 and the medical care platform 60 transmits the new health score to the personal computer of the doctor (e.g. computer) to provide health information of the patient to the doctor for evaluating the recovery situation of the patient.

In accordance with the above description, the intelligent gait analyzing apparatus of the present invention utilizes a first camera to capture a video in which the patient walks, sits down and stands up. Subsequently, the graphic processor acquires the stride, the pace, the humpbacked value, the risk of falling and the time length from the last sitting on the chair then to next standing up based on the video and utilizes the algorithm of gravity center to acquire an original gravity center. At last, the graphic processor calculates the health score by performing a weight function on the stride, the pace, the humpbacked value, the risk of falling and the time length from the last sitting on the chair then to next standing up. Doctor estimates recovery situation and muscle endurance of the patient.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. An intelligent gait analyzing apparatus, comprising:
a first camera capturing a first video in which a patient walks from a starting point to a chair and a second video in which the patient changes a posture from standing up to sitting on the chair; and
an electronic device electrically connected to the first camera to receive the first video and the second video, the electronic device being provided with a graphic processor and a memory storing an algorithm of gravity center, the graphic processor establishing a 3D skeleton model including a plurality of body segments and a moving diagram of the patient based on the first video, the graphic processor acquiring a stride, a pace and a humpbacked value, a risk of falling of the patient based on the moving diagram, the graphic processor utilizing the algorithm of gravity center to acquire an original gravity center based on the moving diagram and acquiring a time length from the last sitting on the chair then to next standing up based on the second video, the graphic processor generating a health score by normalizing and performing a weight function on the stride, the pace, the humpbacked value, the risk of falling and the time length from the last sitting on the chair then to next standing up.

2. The intelligent gait analyzing apparatus according to claim 1, wherein the moving diagram is a dynamic record recoding a motion of the 3D skeleton model corresponding to the patient in the first video and the graphic processor traces a plurality of coordinates corresponding to the plurality of body segments of the 3D skeleton model.

3. The intelligent gait analyzing apparatus according to claim 2, wherein the graphic processor calculates the stride and the pace based on two coordinates corresponding to two feet among the plurality of body segments of the 3D skeleton model in the moving diagram.

4. The intelligent gait analyzing apparatus according to claim 2, wherein the graphic processor utilizes the algorithm of gravity center to calculate the original gravity center based on a moving path of the plurality of coordinates corresponding to the body segments in the moving diagram.

5. The intelligent gait analyzing apparatus according to claim 4, wherein the graphic processor utilizes the correction algorithm to calculate a variation of gravity center based on the original gravity center and the risk of falling of the patient.

6. The intelligent gait analyzing apparatus according to claim 2, further comprising a second camera electrically connected to the electronic device and capturing patient images of the patient, the electronic device creating a point cloud diagram based on the patient images.

7. The intelligent gait analyzing apparatus according to claim 6, wherein the graphic processor acquires a projection distance and a boundary point based on the point cloud diagram and utilizes the algorithm of gravity center to calculate a modified gravity center based on a moving path of the plurality of coordinates of the body segments in the moving diagram, the projection distance and the boundary point.

8. The intelligent gait analyzing apparatus according to claim 2, further comprising a workstation in networked communication with the electronic device and provided with a backup database, the backup database storing a reference 3D skeleton model with a plurality of reference coordinates of body segments.

9. The intelligent gait analyzing apparatus according to claim 8, wherein the workstation transmits the reference 3D skeleton model to the electronic device and the graphic processor utilizes a long short-term memory model or a transformer machine learning model to calculate a variation between the plurality of coordinates and the plurality of reference coordinates or of the health parameters (HP).

10. The intelligent gait analyzing apparatus according to claim 2, further comprising a workstation in networked communication with the electronic device and provided with a history database, the history database storing a history 3D skeleton model with a plurality of history coordinates of body segments.

11. The intelligent gait analyzing apparatus according to claim 10, wherein the workstation transmits the history 3D skeleton model to the electronic device, and the graphic processor utilizes a transformer machine learning model or a long short-term memory model to calculate a similarity score between the plurality of coordinates and the plurality of history coordinates, or of the health parameters (HP) related to abnormal symptom classes from an electric medical record and the graphic processor compares the similarity score with a threshold value.

12. The intelligent gait analyzing apparatus according to claim 1, wherein the electronic device comprises a first database storing the health score and a second database storing the 3D skeleton model and the moving diagram.

13. The intelligent gait analyzing apparatus according to claim 1, wherein the time length from the last sitting on the chair then to next standing up is determined by the graphic processor calculating a time difference for a head or a pelvis of the body segments moving from a first coordinate of the head or the pelvis representing the patient is standing up and a second coordinate of the head or the pelvis representing the patient is sitting on the chair.

14. The intelligent gait analyzing apparatus according to claim 1, wherein the starting point is arranged on a pathway and the pathway is adjacent to the chair to provide the patient to walk from the starting point to the chair.

15. The intelligent gait analyzing apparatus according to claim 1, further comprising a sensor electrically connected to the electronic device and sensing an identification card of the patient to acquire an identification number, the electronic device receiving the identification number.

16. The intelligent gait analyzing apparatus according to claim 15, further comprising a medical care platform in networked communication with the electronic device to receive the identification number and the health score, the medical care platform transmitting an electronic medical record to the electronic device.
